(19) United States
(12) Patent Application Publication
(10) Pub. No.: US 2021/0348153 A1
(43) Pub. Date: Nov. 11, 2021

(54) CELLULOSE-ENCAPSULATED OIL EMULSIONS AND METHODS FOR CELLULASE REGENERATION

(71) Applicant: TECHNION RESEARCH & DEVELOPMENT FOUNDATION LIMITED, Haifa (IL)

(72) Inventors: Dmitry M. REIN, Nesher (IL); Yachin COHEN, Haifa (IL); Gilad ALFASSI, Kiryat Motzkin (IL)

(21) Appl. No.: 17/282,843

(22) PCT Filed: Oct. 3, 2019
(86) PCT No.: PCT/IL2019/051084

§ 371 Date: Apr. 5, 2021

Related U.S. Application Data
(60) Provisional application No. 62/741,055, filed on Oct. 4, 2018.

(51) Int. Cl. C12N 11/04; B01J 13/08; C12N 11/12; C12P 7/10; C12P 19/14; C12N 9/42

(52) U.S. Cl. CPC C12N 11/04; B01J 13/08; C12N 11/12; C12Y 302/01004; C12P 19/14; C12N 9/2437; C12P 7/10

(57) ABSTRACT

Cellulosic capsules comprising an interior hydrophobic medium and use thereof such as for adsorbing cellulose-hydrolyzing enzymes, are provided.

### CROSS REFERENCE TO RELATED APPLICATIONS

This application claims the benefit of priority to U.S. Provisional Patent Application No. 62/741,055 filed Oct. 4, 2018 entitled “CELLULOSE-ENCAPSULATED OIL EMULSIONS AND METHODS FOR CELLULASE REGENERATION”, the contents of which are incorporated herein by reference in their entirety.

### FIELD OF INVENTION

The present invention, in some embodiments thereof, relates to cellulosic capsules and use thereof, for example, for adsorbing cellulose-hydrolyzing enzymes.

### BACKGROUND OF THE INVENTION

Encapsulation is a process in which tiny particles or droplets are surrounded by a coating to impart many useful properties to small capsules. In a relatively simplistic form, a microcapsule is a small sphere with a uniform wall around it. The material inside the microcapsule is referred to as the core, internal phase, or fill, whereas the wall is sometimes called a shell, coating, or membrane. Most microcapsules have diameters between a few tens of nanometers to a few millimeters. The core may be a crystal, a jagged adsorbent particle, an emulsion, a suspension of solids, or a suspension of smaller microcapsules. The microcapsule even may have multiple walls. The reasons for microencapsulation are countless. In some cases, the core must be isolated from its surroundings, as in isolating vitamins from the deteriorating effects of oxygen, retarding evaporation of a volatile core, improving the handling properties of a sticky material, or isolating a reactive core from chemical attack. In other cases, the objective is not to isolate the core completely but to control the rate at which it leaves the microcapsule, as in the controlled release of drugs or pesticides. The problem may be as simple as masking the taste or odor of the core, or as complex as increasing the selectivity of an adsorption or extraction process.

The technique of microencapsulation depends on the physical and chemical properties of the material to be encapsulated: complex coacervation, centrifugal extrusion, vibrational nozzle, spray-drying, interfacial polycondensation, interfacial cross-linking, in-situ polymerization, etc.

Non-food cellulosic biomass is the most abundant renewable bioresource as a collectable, transportable, and storable chemical energy that is far from fully utilized now. The production of biofuels and value-added biochemicals from evenly distributed non-food cellulosic biomass would decrease net greenhouse gas emissions by replacing the use of fossil fuels and would bring benefits to rural economy, national energy security, and the balance of trade.

A significant reduction in the cost of cellulase enzymes, used in standard processes of cellulose hydrolysis, is an urgent requirement to enable the economically sustainable utilization of lignocellulosic materials. The recycling of enzymes from process wastewaters is quite likely the solution to reduce this cost. Considering a generic biorefinery process, for example, production of ethanol from lignocellulose, there are typically three process wastewater streams that are potential enzyme sources in the recycling process: the hydrolysate, the fermentation broth after fermentation, and the stillage (bottom product from the ethanol distillation column).

The main criteria for the suitability of the listed sources for enzymes regeneration are the presence of compounds that inhibit the enzymes in this stream (or the possibility of removal) and the level of stress (physical and chemical) that the enzymes had endured (so as to still remain effective).

Immobilization of enzymes on insoluble solid carriers, that may adsorb modified enzymes in insoluble substrates using relatively large particles (typically 0.1 to 5 μm in diameter), limits their function due to reduced accessibility to the substrate (because of limited openness of the enzymes, which are immobilized in carrier pores) and mass transfer limitations.

The enzyme immobilization is normally performed by their adsorption into carriers or by covalent coupling reactions with amino- or carboxylic acid groups in the enzymes and carriers. An orientation of the coupled enzymes is mostly random, which doubtless poses the risk of decreased or even loss of their activity, for example by blocking of an active site, steric hindrance due to high loading/surface coverage or conformational changes in the enzyme structure.

A large number of different enzymes, required for complete hydrolysis of cellulosic biomass, greatly complicates the choice of effective solid substrate for the immobilizing this array of different enzymes. The high costs associated with the immobilization of the enzymes, loss and destruction during the operation of the immobilizing substrates (carriers) are also great concerns for the applicability of this method.

### SUMMARY OF THE INVENTION

According to one aspect, there is provided a composition comprising at least one capsule comprising a shell and a hydrophobic core, wherein:
(a) a median diameter of the capsule is less than about 3000 nm;
(b) the shell comprises at least one layer comprising cellulosic material, the layer having a thickness of 5 to 50 nm; and
(c) the shell is characterized by an average porosity of 85 to 99%.

According to some embodiments, the cellulosic material is in the form selected from amorphized non-derivatized cellulose, an assemblage of cellulosic particles, and a combination thereof.

According to some embodiments, the shell is characterized by a porosity ranging from 94% to 96%.

According to some embodiments, the weight ratio between the hydrophobic core and the at least one cellulose layer ranges from 30:1 to 1.5:1, respectively.

According to some embodiments, the hydrophobic core comprises one or more materials selected from C2-C10 alkanes, alkenes, alkynes, substituted or non-substituted, and silicones, and any combination thereof.

According to another aspect, there is provided a composition comprising at least one capsule comprising: (i) a shell in the form selected from amorphized non-derivatized cellulose, an assemblage of cellulosic particles, and a combination thereof, (ii) a hydrophobic core, and (iii) one or more hydrolyzing enzymes, wherein:
(a) a median diameter of the capsule is less than about 3000 nm;
(b) the shell comprises at least one layer, wherein the layer having a thickness of 5 to 300 nm;
(c) the hydrolyzing enzymes are adsorbed on the shell.

According to some embodiments, the enzymatic activity of the adsorbed hydrolyzing enzymes in the hydrophilic core medium is less than 20% compared to the enzymatic activity of the corresponding non-adsorbed hydrolyzing enzymes in the hydrophilic medium.

According to some embodiments, the one or more hydrolyzing enzymes are selected from the group consisting of: α-amylase, β-amylase, isoamylase, glucoamylase, pullulanase, cyclodextrin glucano-transferase, β-fructofuranosidase, glucose isomerase, glycoside hydrolase, and combinations thereof.

According to some embodiments, the one or more hydrolyzing enzymes and the shell are present in a weight ratio of 1:50 to 1:5, respectively. According to some embodiments, the weight ratio is 1:40 to 1:30, respectively.

According to another aspect, there is provided a method for inhibiting an activity of one or more cellulose hydrolyzing enzymes in a hydrophilic medium, the method comprising adsorbing the cellulose hydrolyzing enzymes on at least one capsule comprising a shell and a hydrophobic core; wherein:
(i) a median diameter of the capsule is less than about 3000 nm; and wherein
(ii) the shell comprises at least one layer comprising cellulosic material, wherein the layer having a thickness of 5 to 300 nm.

According to another aspect, there is provided a method for recycling one or more cellulose hydrolyzing enzymes into a reaction liquid, the method comprising:
(a) contacting at least a portion of the reaction liquid comprising the cellulose hydrolyzing enzymes to be recycled with at least one capsule comprising a shell and a hydrophobic core;
(b) eliminating the hydrophobic core so as to disintegrate the capsule, thereby desorbing the hydrolyzing enzymes from the shell;
(c) recycling the released enzymes into the reaction liquid;
   wherein (i) a median diameter of the capsule is less than about 3000 nm; and wherein (ii) the shell comprises at least one layer comprising cellulosic material, wherein the layer having a thickness of 5 to 300 nm. According to some embodiments, the thickness ranges from 5 to 50 nm.

According to some embodiments, the method further comprises a step of contacting remnants of the eliminated hydrophobic core with amorphized cellulose, thereby re-forming the capsule.

According to some embodiments, the shell is characterized by an average porosity of 85 to 99%.

According to some embodiments, the cellulosic material is in a form selected from amorphized non-derivatized cellulose, an assemblage of cellulosic particles, and a combination thereof.

According to some embodiments, the step of eliminating the core is performed by evaporating the hydrophobic medium.

According to some embodiments, the released enzymes are characterized by an enzymatic activity of at least 50% of their activity prior to adsorption.

According to some embodiments, the method further comprises a step of hydrolyzing remnants of the layer comprising the amorphized non-derivatized cellulose or the assemblage of cellulosic particles in the reaction liquid.

According to some embodiments, the method is employed in the production of biofuel or biodiesel.

According to some embodiments, the capsule is formed spontaneously in the reaction liquid, simultaneously with the production of biofuel or biodiesel.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents a block-diagram of subunit for enzyme regenerating in a non-limiting exemplary existing biorefinery plant, which uses the disclosed capsules (cellulose-coated trap-emulsions). The dotted-checkered arrows (black and red) indicate the connection points of the disclosed capsules to a functioning plant;

FIG. 2 presents a graph showing the time required for complete adsorption of enzymes depending on the dispersion average capsule size;

FIG. 3 presents a graph showing adsorption of enzymes, depending on the thickness of the capsule's cellulose shell (in the absence of the cellulose enzymatic hydrolysis process);

FIGS. 4A-D present fluorescent microscope images of cellulose-coated oil emulsion at the beginning (FIGS. 4A-B: FIG. 4A—the oil phase only, stained in oil-specific fluorescent pigment, Neil red, and FIG. 4B—the cellulose phase only, stained in cellulose-specific fluorescent green pigment, Calcofluor-white) and at the beginning of the enzymatic cellulose hydrolysis process (FIG. 4C): the “trap”-emulsion formation (the capsules, comprising both oil and cellulose), showing a visible uniform cellulose casting and smooth boundary between the cellulosic shell and oil core, and at the and at the end of the enzymatic cellulose hydrolysis process (FIG. 4D), i.e. upon the evaporation of the oil; and

FIGS. 5A-C presents fluorescent microscope images of the disclosed capsules “trap”-oil (hexane) emulsion, saturated with captured enzymes (FIG. 5A, arrow). Cellulose is colored by cellulose-specific fluorescent pigment (FIG. 5A, green color); oil—by the oil-specific fluorescent pigment (FIG. 5B, red color); FIG. 5A—cellulose; FIG. 5B—oil; FIG. 5C—joint florescence. Scale bar is 2 μm.

### DETAILED DESCRIPTION OF THE INVENTION

The present invention, in some embodiments thereof, relates to cellulosic particles, for example, in the form of capsules, comprising interior hydrophobic medium and uses the same, for example, for adsorbing cellulose hydrolyzing enzymes.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

### Capsules

According to some embodiments, there is provided a composition comprising at least one capsule having a shell and a hydrophobic core, wherein: the capsule has a median diameter of is less than about 3000 nm, and the shell comprises a cellulose having a thickness of 5 to 500 nm.

According to some embodiments, there is provided a composition comprising at least one capsule having a shell and a hydrophobic core, wherein: the capsule has a median diameter of is less than about 3000 nm, and the shell comprises a cellulosic material having a thickness of 5 nm to 500 nm.

In some embodiments, the core consists of, or comprises an interior hydrophobic space or medium encapsulated in non-derivatized cellulose, wherein, in some embodiments, the cellulose is surrounded by a hydrophilic medium.

In some embodiments, the shell is in the form of at least one layer comprising or consisting of non-derivatized cellulose.

In some embodiments, by cellulosic material it is meant to refer to a pure non-derivatized cellulose hydrogel.

In some embodiments, the term “cellulosic material” refers to derivatized cellulose, e.g., cellulose acetate as defined hereinbelow.

In some embodiments, the cellulosic material is purified cellulose. In some embodiments, the cellulosic material is non-derivatized cellulose.

The term “derivatized cellulose” may denote a product in which hydroxyl groups of a cellulose product are partially or fully reacted with various reagents to afford derivatives. Cellulose esters and cellulose ethers are further non-limiting examples for such materials. More particularly, cellulose derivatives include ethyl cellulose, methyl cellulose, cellulose acetates e.g., secondary cellulose acetates (partially acetylated cellulose), cellulose triacetate, hydroxyalkylated cellulose, e.g., hydroxypropyl cellulose, carboxymethyl cellulose, hydroxypropylmethyl cellulose, hydroxyethyl methyl cellulose, etc.

In some embodiments, the cellulosic material has a non-crystalline, amorphized structure.

The term “crystalline” as used herein, refers to a structure having three dimensional ordered arrangement of atoms or molecules, which possesses symmetry characteristics.

The term “amorphous” or any grammatical inflection thereof, as used herein refers to the lack of regular internally ordered arrangement, or the antithetical form of the crystalline form. In a further embodiment, the composition is in the form of a capsule comprising a shell and a core. In a further embodiment, the capsule is 10 nm to 5000 nm in diameter.

In a further embodiment, a thickness of the shell thickness ranges from about 0.1% to about 500% of the core.

In a further embodiment, cellulosic material is an emulsifying agent.

In some embodiments, the layer is a continuous molecular layer. In some embodiments, the layer comprises or is in the form of a group of particles. In some embodiments, the layer comprises or is in the form of a combination of a continuous molecular layer and a group of particles. The term “layer” may refer to one or more layers.

The term “continuous layer” is intended principally to cover the situation when adjacent cellulose molecules or cellulose particles and/or cellulose hydrogel droplets relics spread out and join together to form a thin uninterrupted layer but is not intended to exclude either layers with occasional voids or layers. The particles may be connected or separated.

In some embodiments, the layer comprises an assemblage of cellulosic particles.

The terms “assemblage”, “mat” or “aggregate”, which may be used herein interchangeably refer to any configuration of a mass of intertwined individuals, or, in some embodiments to discrete particles.

The term “capsule” refers to either a rigid, hard or soft core-shell particle that, in some embodiments serves as a vehicle for hydrophobic liquids or semi-solids.

The capsule may be generally shaped as a sphere, a rod, a cylinder, a ribbon, a sponge, and any other shape, or can be in a form of a cluster of any of these shapes, or can comprise a mixture of one or more shapes.

In some embodiments, at least some, and in some embodiments, most of the capsules are generally shaped as spheres.

The term “core” refers to the central region (typically the hydrophobic domain(s)) of the structure, which typically contains a closed hollow.

As used herein, the terms “shell”, or “coating” which are used hereinthroughout interchangeably, refer to the sphere (typically the hydrophilic domain(s)) surrounding the core. The term “sphere” is used only for the purpose of illustration and it is to be construed that is not only limited to spherical shape but also includes any shape which may find suitability to at least some embodiments of the present invention.

The term “closed”, as used herein, is a relative term with respect to the size, the shape and the composition of two entities, namely an entity that defines an enclosure (the enclosing entity) and the entity that is being at least partially enclosed therein. In general, the term “closed” refers to a morphological state of an object which has discrete inner medium and outer surface(s) or layer(s) which are substantially disconnected, wherein the inner surface constitutes the boundary of the enclosed area or space. The enclosed area or space may be secluded from the exterior area of space which is bounded only by the outer surface (the shell).

In the context of the present invention, the closure of the enclosing entity may depend of the size, shape and chemical composition of the entity that is being enclosed therein, such that the enclosing entity may be “closed” for one entity and at the same time be “open” for another entity. For example, structures presented herein are closed with respect to certain chemical entities which cannot pass through their enclosing shell, while the same “closed” structures are not closed with respect to other entities.

In the context of the present invention, the same “closed” structures may be affected by certain conditions e.g., pH, temperature, concentration of the hydrophobic medium within the core, thickness of the cellulosic layer, etc.

In some embodiments, the capsule has a median diameter of 10 nm to 3000 nm, 20 nm to 3000 nm, 40 nm to 3000 nm, 50 nm to 3000 nm, 80 nm to 3000 nm, 100 nm to 3000 nm, 150 nm to 3000 nm, 300 nm to 3000 nm, 500 nm to 3000 nm, 1000 nm to 3000 nm, 1500 nm to 3000 nm, 2000 nm to 3000 nm, 10 nm to 2800 nm, 20 nm to 2800 nm, 40 nm to 2800 nm, 50 nm to 2800 nm, 80 nm to 2800 nm, 100 nm to 2800 nm, 150 nm to 2800 nm, 300 nm to 2800 nm, 500 nm to 2800 nm, 1000 nm to 2800 nm, 1500 nm to 2800 nm, 2000 nm to 2800 nm, 10 nm to 2500 nm, 20 nm to 2500 nm, 40 nm to 2500 nm, 50 nm to 2500 nm, 80 nm to 2500 nm, 100 nm to 2500 nm, 150 nm to 2500 nm, 300 nm to 2500 nm, 500 nm to 2500 nm, 1000 nm to 2500 nm, 1500 nm to 2500 nm, 2000 nm to 2500 nm, 10 nm to 2000 nm, 20 nm to 2000 nm, 40 nm to 2000 nm, 50 nm to 2000 nm, 80 nm to 2000 nm, 100 nm to 2000 nm, 150 nm to 2000 nm, 300 nm to 2000 nm, 500 nm to 2000 nm, 1000 nm to 2000 nm, 1500 nm to 2000 nm, 10 nm to 1000 nm, 20 nm to 1000 nm, 40 nm to 1000 nm, 50 nm to 1000 nm, 80 nm to 1000 nm, 100 nm to 1000 nm, 150 nm to 1000 nm, 300 nm to 1000 nm, 500 nm to 1000 nm, 10 nm to 800 nm, 20 nm to 800 nm, 40 nm to 800 nm, 50 nm to 800 nm, 80 nm to 800 nm, 100 nm to 800 nm, 150 nm to 800 nm, 300 nm to 800 nm, or 500 nm to 800 nm, including any range therebetween.

In some embodiments, the term “capsule” is also meant to refer to a plurality of capsules.

In some embodiments, the plurality of capsules has a median size of about 2900 nm, about 2800 nm, about 2700 nm, about 2600 nm, about 2500 nm, about 2400 nm, about 2300 nm, about 2200 nm, about 2100 nm, about 2000 nm, about 1900 nm, about 1800 nm, about 1700 nm, about 1600 nm, about 1500 nm, about 1400 nm, about 1300 nm, about 1200 nm, about 1100 nm, about 1000 nm, about 900 nm, about 800 nm, about 700 nm, about 600 nm, about 500 nm, about 400 nm, about 300 nm, about 200 nm, about 100 nm, or about 50 nm, including any value and range therebetween.

In some embodiments, the plurality of capsules has a median size of 10 nm to 3000 nm, 20 nm to 3000 nm, 40 nm to 3000 nm, 50 nm to 3000 nm, 80 nm to 3000 nm, 100 nm to 3000 nm, 150 nm to 3000 nm, 300 nm to 3000 nm, 500 nm to 3000 nm, 1000 nm to 3000 nm, 1500 nm to 3000 nm, 2000 nm to 3000 nm, 10 nm to 2800 nm, 20 nm to 2800 nm, 40 nm to 2800 nm, 50 nm to 2800 nm, 80 nm to 2800 nm, 100 nm to 2800 nm, 150 nm to 2800 nm, 300 nm to 2800 nm, 500 nm to 2800 nm, 1000 nm to 2800 nm, 1500 nm to 2800 nm, 2000 nm to 2800 nm, 10 nm to 2500 nm, 20 nm to 2500 nm, 40 nm to 2500 nm, 50 nm to 2500 nm, 80 nm to 2500 nm, 100 nm to 2500 nm, 150 nm to 2500 nm, 300 nm to 2500 nm, 500 nm to 2500 nm, 1000 nm to 2500 nm, 1500 nm to 2500 nm, 2000 nm to 2500 nm, 10 nm to 2000 nm, 20 nm to 2000 nm, 40 nm to 2000 nm, 50 nm to 2000 nm, 80 nm to 2000 nm, 100 nm to 2000 nm, 150 nm to 2000 nm, 300 nm to 2000 nm, 500 nm to 2000 nm, 1000 nm to 2000 nm, 1500 nm to 2000 nm, 10 nm to 1000 nm, 20 nm to 1000 nm, 40 nm to 1000 nm, 50 nm to 1000 nm, 80 nm to 1000 nm, 100 nm to 1000 nm, 150 nm to 1000 nm, 300 nm to 1000 nm, 500 nm to 1000 nm, 10 nm to 800 nm, 20 nm to 800 nm, 40 nm to 800 nm, 50 nm to 800 nm, 80 nm to 800 nm, 100 nm to 800 nm, 150 nm to 800 nm, 300 nm to 800 nm, or 500 nm to 800 nm, including any range therebetween.

In some embodiments, the term “size” refer to diameter.

In some embodiments, the median size (e.g., diameter) ranges from about 10 nm to 500 nm. In some embodiments, the median size ranges from about 10 nm to about 300 nm. In some embodiments, the median size ranges from about 5 nm to about 200 nm. In some embodiments, the median size ranges from about 5 nm to about 100 nm. In some embodiments, the median size ranges from about 1 nm to 50 nm, and in some embodiments, it is lower than 35 nm.

In some embodiments, the shell has a thickness (also referred to as: “shell thickness”) of 5 to 300 nm, 5 to 200 nm, 5 to 100 nm, or 5 to 50 nm.

In some embodiments, the shell has a thickness of 5 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 250 nm, 255 nm, 260 nm, 265 nm, 270 nm, 275 nm, 280 nm, 285 nm, 290 nm, 295 nm, or 300 nm, including any value and range therebetween.

In some embodiments, the shell has a thickness of 5 nm to 500 nm, 10 nm to 500 nm, 15 nm to 500 nm, 30 nm to 500 nm, 50 nm to 500 nm, 80 nm to 500 nm, 90 nm to 500 nm, 100 nm to 500 nm, 5 nm to 450 nm, 10 nm to 450 nm, 15 nm to 450 nm, 30 nm to 450 nm, 50 nm to 450 nm, 80 nm to 450 nm, 90 nm to 450 nm, 100 nm to 450 nm, 5 nm to 300 nm, 10 nm to 300 nm, 15 nm to 300 nm, 30 nm to 300 nm, 50 nm to 300 nm, 80 nm to 300 nm, 90 nm to 300 nm, 100 nm to 300 nm, 5 nm to 200 nm, 10 nm to 200 nm, 15 nm to 200 nm, 30 nm to 200 nm, 50 nm to 200 nm, 80 nm to 200 nm, 90 nm to 200 nm, 100 nm to 200 nm, 5 nm to 150 nm, 10 nm to 150 nm, 15 nm to 150 nm, 30 nm to 150 nm, 50 nm to 150 nm, 80 nm to 150 nm, 90 nm to 150 nm, 100 nm to 150 nm, 5 nm to 100 nm, 10 nm to 100 nm, 15 nm to 100 nm, 30 nm to 100 nm, 50 nm to 100 nm, or 80 nm to 100 nm, including any range therebetween.

The term “shell thickness” is used to mean the cross-sectional capsule wall thickness of the capsule. In general, to measure the cross-sectional capsule wall thickness, the shell may be cut along their minor axis, or, in some embodiments, may be cut in any direction through the center. The measurement may be accomplished by using the cross-section of a single capsule to obtain the shell thickness reflecting the whole cross-section. For example, two points with maximum and minimum thicknesses may be measured and averaged.

In some embodiments, the cross-sectional capsule wall thickness may be measured by a microscopic technique.

In some embodiments, the shell thickness ranges from about 0.1% to about 500% of their inner (core) part. In some embodiments, the shell thickness ranges from about 1% to about 500% of their core part. In some embodiments, the shell thickness ranges from about 0.1% to about 100% of their core part. In some embodiments, the shell thickness ranges from about 0.5% to about 400% of their core part. In some embodiments, the shell thickness ranges from about 1% to about 300% of their core part. In some embodiments, the shell thickness ranges from about 10% to about 200% of their core part. In some embodiments, the shell thickness ranges from about 50% to about 300% of their core part.

In some embodiment, the shell is characterized by a defined porous structure, or porosity.

The term “porous” as used herein refers to a shell (or a layer therein) that comprises pores, holes, or voids.

The term “porosity” refers to a measure of the void spaces in the particle, and is measured as a fraction, between 0-1, or as a percentage between 0 to 100%.

In some embodiments, the shell is characterized by an average porosity of at least 80%, least 85%, at least 90%, at least 95%, or at least 99%. In some embodiments, the shell is characterized by an average porosity of 90% to 99%, or, in some embodiments, 94% to 96%. In some embodiments, the shell is characterized by an average porosity of 85%, 90%, 95%, or 99%, including any value and range therebetween. In some embodiments, the shell is characterized by an average porosity of 80% to 99%, 85% to 99%, 89% to 99%, 90% to 99%, 92% to 99%, 80% to 95%, 85% to 95%, 89% to 95%, 90% to 95%, 92% to 95%, 80% to 92%, 85% to 92%, or 89% to 92%, including any range therebetween.

In some embodiments, at least a portion of the shell is characterized by a porosity of at least 80%, least 85%, at least 90%, at least 95%, or at least 99%. In some embodiments, the shell is characterized by a porosity of 90% to 99%, or, in some embodiments, 94% to 96%. In some embodiments, the shell is characterized by a porosity of 85%, 90%, 95%, or 99%, including any value and range therebetween.

By “a portion” it is meant to refer to, for example, an outer surface or a volume or a part thereof. In some embodiments, by “a portion” as used herein throughout, it is meant e.g., at least 1 percent, at least 20 percent, at least 30 percent, at least 40 percent, at least 50 percent, at least 60 percent, at least 70 percent, at least 80 percent, at least 90 percent, and optionally all of the surface is characterized by a porosity of 85% to 99%, 90% to 99%, or, in some embodiments, 94% to 96%.

In some embodiments, the core comprises a hydrophobic medium.

In some embodiments, the disclosed capsules are dispersed in a hydrophilic medium, thereby forming a dispersion or an emulsion.

The term “hydrophobic”, in some embodiments, refers to molecules that are typically free of polar groups. The term “hydrophilic”, in some embodiments, refers to molecules that typically have polar groups. The terms “hydrophobic medium”, “oil medium”, or “oil” may be used hereinthroughout interchangeably.

In some embodiments, the boundary between hydrophobicity and hydrophilicity occurs when the difference between the apolar attraction and the polar repulsion between molecules or particles of material immersed in water is equal to the cohesive polar attraction between the water molecules. Under these conditions, the interfacial free energy of interaction between particles of a material, immersed in water (ignoring electrostatic interactions), ΔG^{IF}, is exactly zero. When the ΔG^{IF} is positive, the interaction of the material with water dominates and the surface of the material is hydrophilic; when ΔG^{IF} is negative, the polar cohesive attraction between the water molecules dominates and the material is hydrophobic. Thus, the sign of ΔG^{IF} defines the nature of the surface and the magnitude of ΔG^{IF} and is used, in some embodiments, as the natural quantitative measure of the surface hydrophobicity or hydrophilicity.

In some embodiments of the invention, cellulose molecules are ordered in the boundaries of the hydrophobic medium or space and water. Without being bound by any particular theory, it is assumed that the stabilization of the oil/water emulsion is affected by the presence of cellulose molecules in the boundaries of oil/water.

In some embodiments, an interior hydrophobic space is the core or the nucleus of a capsule of the invention which comprises a hydrophobic material such as, but not limited to, hydrocarbon. In some embodiments, an interior hydrophobic space comprises hydrophobic interacting groups of non-derivatized cellulose.

In some embodiments, non-derivatized cellulose comprises up to 40% impurities. In some embodiments, non-derivatized cellulose comprises up to 35% impurities. In some embodiments, non-derivatized cellulose comprises up to 30% impurities. In some embodiments, non-derivatized cellulose comprises up to 25% impurities. In some embodiments, non-derivatized cellulose comprises up to 20% impurities. In some embodiments, the impurities comprise organic impurities. In some embodiments, impurities include: lignin, hemicellulose, etc.

As described herein, in some embodiments, the disclosed capsules are dispersed in a hydrophilic medium, e.g., an aqueous solution.

In some embodiments, the hydrophilic medium is the solution which surrounds the capsule(s) of the invention. In some embodiments, the medium is the solution which may interact with the outer surface of a capsule of the invention.

In some embodiments, the hydrophobic core (also referred to as: “interior hydrophobic space”) comprises or consists of hydrophobic solid particles or liquid volatiles.

Non-limiting exemplary liquid volatiles are selected from C2-C10 alkanes (e.g., pentane, hexane), alkenes or alkynes, substituted or non-substituted, silicones, and a liquefied household gas.

In some embodiments, the hydrophobic core comprises an insoluble frozen gas or insoluble gas (e.g., in the form of bubbles) which are poorly or slowly soluble in water.

The term “insoluble gas” as used herein intends to include gases and mixtures of gases which are entirely insoluble, as well as mixtures of gases which contain minor amounts (less than 20% v/v) of soluble gas(es) such as air.

The terms “dispersed”, “dispersion”, or any grammatical inflection thereof used herein refer to a continuous phase distribution, or to an emulsion, of the disclosed capsules throughout a material, a medium, e.g., the hydrophilic medium.

The term “emulsion” used herein to denote a system having at least two liquid phases, one of which is dispersed in the other.

The different types of emulsions may be defined by reference to the type of liquid forming the outer phase vs. the type of liquid forming the dispersed phase. In this connection, when an oil phase is dispersed in a water phase, the emulsion is terms “oil-in-water emulsion” or the “normal emulsion”.

In some embodiments, the composition consists or comprises capsules in a hydrophilic medium, wherein the capsules have an interior hydrophobic space separated from the hydrophobic medium by at least one layer consisting cellulose. In some embodiments, the composition described herein is devoid of a surfactant. In some embodiments, the composition described herein is oil-in-water composition or emulsion.

In some embodiments, the cellulosic material is, or is capable of being in the form of hydrogel.

The term “hydrogel” as used herein refers to a network of natural or synthetic polymer chains capable to contain water.

The terms “cellulosic material” refers to a material that contains cellulose, typically, but not exclusively, materials derived from plant sources that contain cellulose. Cellulose may comprise a linear polysaccharide polymer composed of β-1,4 linked D-glucose molecules.

In some embodiments, the weight ratio between the hydrophobic core and the cellulose layer ranges from 30:1 to 1.5:1, respectively. In some embodiments, the weight ratio between the hydrophobic core and the cellulose layer ranges from 25:1 to 1.5:1, respectively. In some embodiments, the weight ratio between the hydrophobic core and the cellulose layer ranges from 20:1 to 1.5:1, respectively. In some embodiments, the weight ratio between the hydrophobic core and the cellulose layer ranges from 20:1 to 1:1, respectively. In some embodiments, the weight ratio between the hydrophobic core and the cellulose layer ranges from 15:1 to 1.5:1, respectively.

In some embodiments, the weight ratio between the hydrophobic core and the cellulose layer is 30:1, 25:1, 20:1, 15:1, 10:1, 5:1, 4:1, 3:1, 2:1, or 1.5:1, respectively, including any value and range therebetween.

A Capsule-Enzyme Composition

According to some embodiments, there is provided a composition comprising at least one capsule having a shell and a hydrophobic core, wherein: the capsule has a median diameter of is less than about 3000 nm; and the shell comprises a cellulosic material, having a thickness of 5 to 500 nm, and wherein the composition further comprises one or more cellulose hydrolyzing enzymes attached to the shell. In some embodiments, the shell comprises cellulose. In some embodiments, the shell has a thickness of 5 nm to 300 nm.

Embodiments of “capsule”, “shell”, “hydrophobic core”, “thickness” are described hereinabove under “A Capsule”.

In some embodiments, the composition comprises a hydrophilic medium, such that the capsules are dispersed in the hydrophilic medium, as described hereinabove.

In some embodiments, by “attached to” it is meant that the enzymes are adsorbed on the hydrophobic face of cellulose layer. In some embodiments, by “adsorbed” (also referred to as “loaded” or “captured”) it is meant physically adsorbed.

In some embodiments, enzymes are preferentially adsorbed on the hydrophobic face of cellulose or onto hydrophobic glucan planes of cellulose molecules.

In some embodiments, enzymes are adsorbed on the outer surface of the cellulosic layer. In some embodiments, by “outer surface” it is meant to refer to the surface exposed the hydrophilic medium.

In some embodiments, adsorbing the enzymes is reversible (i.e. the enzymes may be released or desorbed from the shell of the capsule) upon applying certain conditions as described below.

In some embodiments, the hydrolyzing enzymes are selected from, without being limited thereto, α-amylase, β-amylase, β-glucosidase, endo-cellulase, exo-cellulase, isoamylase, glucoamylase, pullulanase, cyclodextrin glucano-transferase, β-fructofuranosidase, glucose isomerase, glycoside hydrolase and any combination thereof.

In some embodiments, the enzyme is selected from lipases.

In some embodiments, the lipases are selected from, without being limited thereto, Ryzopus oryzae, Rhizomucor miehei, Mucor miehei, Pseudomonas fluorescens, Mucor javanicus, Candida rugosa and Rhizopus niveus, as well as lipase OF from Candida cylindracea (C. rugosa) and lipase QLC from Alcaligenes sp. and a combination thereof.

In some embodiments, cellulose capsules of the invention dispersed in an aqueous media are loaded with cellulose hydrolyzing enzymes, wherein, and without being bound by any particular theory, the loading degree is dictated (e.g., increased) by at least one factor selected from, the following factors, without being limited thereto: (i) absence of, or low crystallinity in cellulose shell, (ii) exceedingly small cellulose shell thickness (e.g., less than 500 nm), (iii) cellulose shell's porosity as disclosed herein and (iv) cellulose shell specific surface area (e.g., more than 600 m²/g).

In some embodiments, the enzyme and the cellulose layer are present in a weight ratio of 1:10,000 to 1:5, respectively. In some embodiments, the enzyme and the cellulose layer are present in a weight ratio of 1:1,000 to 1:5, respectively. In some embodiments, the enzyme and the cellulose layer are present in a weight ratio of 1:1,000 to 1:10, respectively. In some embodiments, the enzyme and the cellulose layer are present in a weight ratio of 1:100 to 1:10, respectively. In some embodiments, the enzyme and the cellulose layer are present in a weight ratio of 1:50 to 1:5, respectively. In some embodiments, the enzyme and the cellulose layer are present in a weight ratio of 1:50 to 1:10, respectively.

In some embodiments, the enzyme and the cellulose layer are present in a weight ratio of 1:40 to 1:30, respectively. In some embodiments, the enzyme and the cellulose layer are present in a weight ratio of 1:40, 1:39, 1:38, 1:37, 1:36, 1:35, 1:34, 1:33, 1:32, 1:31, or 1:30, respectively, including any value and range therebetween.

In some embodiments, the hydrolyzing enzyme, upon adsorbing to the cellulosic layer of the disclosed capsule, is characterized by enzymatic activity of less than 80%, less than 70%, less than 60%, less than 50%, less than 40%, less than 30%, less than 20%, or less than 10%, of the enzyme activity in a comparable situation lacking the attachment of the enzyme to the capsule in the same hydrophilic medium. Methods for determining a level of a catalytic activity of an enzyme are known in the art.

In some embodiments, the hydrolyzing enzyme, upon adsorbing to the cellulosic layer of the disclosed capsule, is characterized by enzymatic activity of 1% to 80%, 5% to 80%, 10% to 80%, 20% to 80%, 30% to 80%, 1% to 70%, 5% to 70%, 10% to 70%, 20% to 70%, 30% to 70%, 1% to 50%, 5% to 50%, 10% to 50%, 20% to 50%, 30% to 50%, 1% to 40%, 5% to 40%, 10% to 40%, 20% to 40%, 1% to 20%, 5% to 20%, 10% to 20%, 1% to 15%, 5% to 15%, or 10% to 15%, of the enzyme activity in a comparable situation lacking the attachment of the enzyme to the capsule in the same hydrophilic medium.

In some embodiments, the hydrolyzing enzyme, upon desorbing from the cellulosic layer of the disclosed capsule, is characterized by an enzymatic activity of at least 50%, at least 60%, at least 70%, at least 80%, or at least 90% of the initial enzyme activity, i.e. prior to adsorbing of the enzyme to the disclosed capsule.

In some embodiments, the hydrolyzing enzyme, upon desorbing from the cellulosic layer of the disclosed capsule, is characterized by an enzymatic activity of 50% to 99%, 60% to 99%, 70% to 99%, 80% to 99%, 85% to 99%, 89% to 99%, 90% to 99%, 50% to 96%, 60% to 96%, 70% to 96%, 80% to 96%, 85% to 96%, 89% to 96%, 90% to 96%, 50% to 93%, 60% to 93%, 70% to 93%, 80% to 93%, 85% to 93%, 89% to 93%, 50% to 90%, 60% to 90%, 70% to 90%, 80% to 90%, 85% to 90%, or 89% to 90%, of the initial enzyme activity, i.e. prior to adsorbing of the enzyme to the disclosed capsule.

Accordingly, in some embodiments, there is provided a method for inhibiting an activity of cellulose hydrolyzing enzyme(s) in a medium, the method comprising contacting the hydrolyzing enzyme(s) with the disclosed capsule(s). In some embodiments, the medium is a hydrophilic medium.

### Recycling Methods

According to an aspect of some embodiments of the present invention, there is provided a method for removing a cellulose hydrolyzing enzymes (also referred to as “trap-emulsion method) from a reaction liquid, the method comprising: (i) contacting at least a portion of the reaction liquid comprising the cellulose hydrolyzing enzymes to be recovered with one or more of the herein disclosed capsule in an embodiment thereof, thereby adsorbing the cellulose hydrolyzing enzymes on a shell of the capsule. In some embodiments the method further comprises a subsequent step of (ii) eliminating the hydrophobic medium of the core of the capsule, thereby desorbing (releasing to the medium) the hydrolyzing enzymes from the shell of the capsule.

In some embodiments, the reaction liquid comprises oil/water volume ratio which may be set, for example, to 1:6, 1:5, 1:4, 1:3, 1:2, 1:1, 2:1, 3:1, 4:1, 5:1, or 6:1, respectively, including any value therebetween. In some embodiments, the reaction liquid comprises wastewater.

In some embodiments, the reaction medium comprises oil/ethanol volume ratio set to 1:6, 1:5, 1:4, 1:3, 1:2, 1:1, 2:1, 3:1, 4:1, 5:1, or 6:1, respectively, including any value therebetween.

In some embodiments, the desorbed (also referred to as “released”) enzymes (in step (ii)) is obtained in a hydrophilic (e.g., aqueous) solution.

Therefore, in some embodiments, the method further comprises a step of recycling the enzymes released in step (ii) into the reaction liquid. This may provide a further economic and environmental benefit that the hydrophilic (e.g., aqueous) solution used to recover the enzyme may be recycled, for example, for use in adsorbing further enzymes.

In some embodiments, upon desorbing the hydrolyzing enzymes from the shell of the capsule, the enzymatic activity of the enzyme is recovered.

Herein, by “recovered” it is meant that at least 50%, at least 60%, at least 70%, at least 80%, or at least 90% of the enzyme activity is restored with respect to their initial activity, i.e. prior the attachment (adsorbing) of the enzyme to the capsule.

In some embodiments, the enzymatic activity of the enzyme is maintained after successive recycling rounds. In some embodiments, the enzymatic activity of the enzyme is maintained after 1 recycling rounds, 2 recycling rounds, 3 recycling rounds, 4 recycling rounds, or 5 recycling rounds. In some embodiments, the enzymatic activity of the enzyme is maintained after 1 cycle, 2 cycles, 3 cycles, 4 cycles, or 5 cycles of adsorbing and desorbing the enzymes from the shell of the capsule.

As used herein, by the term “maintained” is meant that at least 35%, at least 38%, at least 40%, at least 50%, at least 60%, at least 70%, at least 80%, or at least 90% of the enzyme activity is restored with respect to their initial activity, i.e. prior the attachment (adsorbing) of the enzyme to the capsule.

In some embodiments, the released enzymes are characterized by an enzymatic activity of 50% to 99%, 60% to 99%, 70% to 99%, 80% to 99%, 85% to 99%, 89% to 99%, 90% to 99%, 50% to 96%, 60% to 96%, 70% to 96%, 80% to 96%, 85% to 96%, 89% to 96%, 90% to 96%, 50% to 93%, 60% to 93%, 70% to 93%, 80% to 93%, 85% to 93%, 89% to 93%, 50% to 90%, 60% to 90%, 70% to 90%, 80% to 90%, 85% to 90%, or 89% to 90%, of their activity prior to adsorption.

In some embodiments, the method may further includes a step in which after the step of releasing the enzymes and prior to the recycling step, the hydrophilic medium (e.g., the aqueous solution) is subjected to a filtration step to provide a concentrated enzyme solution and e.g., water.

In some embodiments, by “recycling”, or any grammatical inflection thereof, it is meant that at least 30%, at least 40%, at least 50%, at least 60%, at least 70%, at least 80%, or at least 90%, of the adsorbed enzymes are recycled to the reaction liquid.

Herein, the step of eliminating the hydrophobic medium is assisted by one or more means selected from, without being limited thereto, evaporation, melting, centrifugation, and adsorption of the hydrophobic medium.

Therefore, in some embodiments, the removal of the internal core of the capsule can be performed both by means of centrifugation, melting and by means of evaporation of this core, in a condition according to the composition.

In some embodiments, the adsorption and the recycling processes are each organized in such a way that they do not affect the properties of the enzymes being processed.

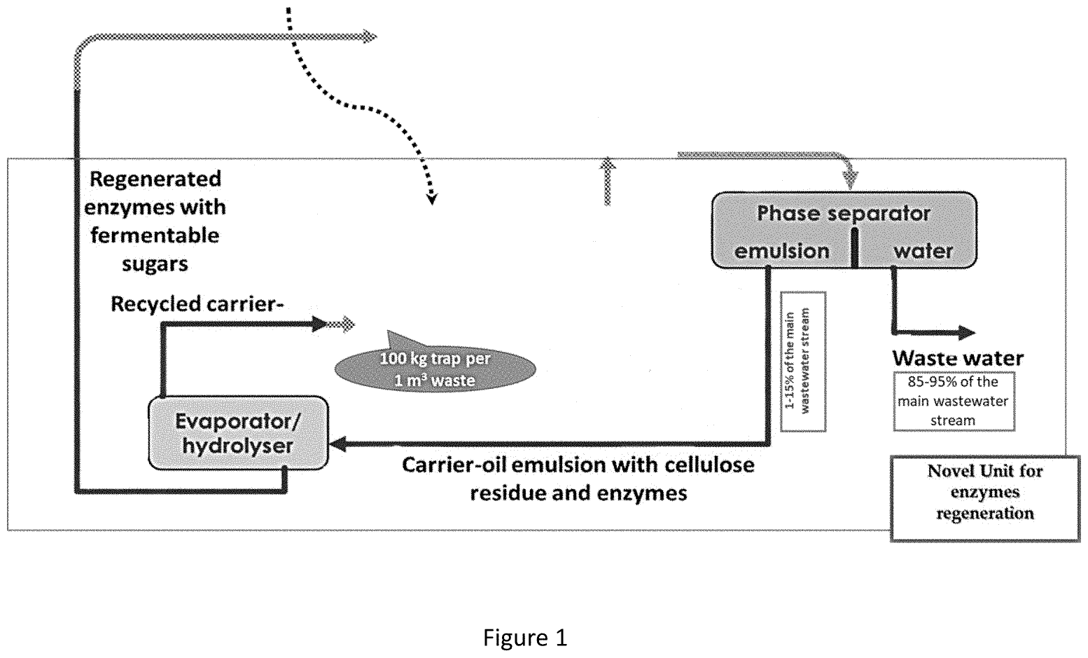

Reference is made to FIG. 1 presenting a block-diagram showing a non-limiting exemplary process utilizing the disclosed trap-emulsion method to recover cellulose hydrolysis enzymes from process waste stream and the functionality of the recovered enzymes.

In some embodiments, the evaporation step of the volatile hydrophobic substance constituting the core is performed under mild conditions (e.g., at temperature 40-50° C. to avoid denaturation of the enzymes).

In some embodiments, the disclosed method is conjugated to a cellulose biofuel manufacturing stream. That is, in some embodiments, the resulting aqueous solution of active regenerated enzymes, optionally, with a small amount of dissolved fermentable sugars (which appear after cellulose shells' hydrolysis at a previous process stage) is directly returned to the main biofuel manufacturing process.

Typically, but not exclusively, for maintaining a biofuel production rate that varies within less than |±15%|, the disclosed method enables recycling of about 1% to 15%, e.g., not more than 1%, 2%, 3%, 4%, 5%, 6%, 7%, 8%, 9%, 10%, 11%, 12%, 13%, 14%, or 15%, by volume, including any value and range therebetween, of the main wastewater stream exiting from the initial reaction liquid.

In some embodiments, prior to the recycling step, the releasing of the adsorbed enzymes into the hydrophilic (e.g., aqueous) surrounding medium is accompanied by hydrolyzing cellulose residues of the former emulsion shell by desorbed enzymes.

Hence, in some embodiments, following eliminating (e.g., evaporating) the interior hydrophobic substance of the disclosed capsules, the capsules structure become disintegrated and fragmented, with at least 70%, at least 80% or at least 90% of the enzymes being desorbed and released to the surrounding medium, and capable of hydrolyzing at least part of the shell residues. In some embodiments components formed and/or separated during this process (enzymes, glucose and the capsule's interior hydrophobic substance) are returned to the standard biofuel processes.

In some embodiments, the biofuel is selected from the group consisting of an alcohol, an alkene, and an alkane. In some embodiments, the alcohol is selected from the group consisting of methanol, ethanol, propanol, isobutanol and n-butanol, or a combination thereof.

In some embodiments, the biofuel is derived from a cellulosic material. In some embodiments, the biofuel is derived from starch. In some embodiments, the disclosed capsule is formed directly or spontaneously in the reaction liquid comprising aqueous solution and oil.

By “simultaneously” it is meant that the disclosed capsule may be formed together with the production of biofuel or biodiesel. In these embodiments, some of the pretreated cellulose present in the main production stream for biofuel production may be used to produce the disclosed capsule.

In some embodiments, biofuel produced (e.g., ethanol) is subsequently fermented. In some embodiments, the bio-fuel is allowed to react with the oil medium, thereby forming biodiesel.

By “fermented”, or any grammatical inflection thereof, it is meant to refer to a chemical change induced in a saccharide substance by the action of an enzyme, whereby the substance is split into simpler compounds. Specifically, the term “fermentation” includes the anaerobic dissimilation of substrates with the production of energy and reduced compounds, the final products thereof being organic acids, alcohols, such as, without being limited thereto, methanol, ethanol, isopropanol, butanol, etc., and CO₂. Such products, are typically secreted and are fermentation resultant of either microbial or yeast fermentation.

In some embodiments, the biofuel is recovered. Recovery of biofuels may be recovered according to methods known in the art. Alcohols such as ethanol, methanol, and/or butanol may be recovered from liquid material by molecular sieves, distillation, and/or other separation techniques. For example, ethanol can be concentrated by fractional distillation to about 90% or about 95% by weight. There are several methods available to further purify ethanol beyond the limits of distillation, and these include, but are not limited to, drying (e.g., with calcium oxide or rocksalt), the addition of small quantities of benzene or cyclohexane, molecular sieve, membrane, or by pressure reduction.

Other ways of collecting biofuel products include e.g., centrifugation, temperature fractionalization, chromatographic methods and electrophoretic methods.

In some embodiments, the alcoholic compound is extracted into oily phase. In some embodiments, the alcoholic compound is extracted by assistance of vacuum.

The purified biofuels products may be stoked in a separate container(s).

In some embodiments, solvent is at least partially removed from the emulsion.

There are several techniques available for removing a solvent (or solvent combination) from an emulsion, as known to those versed in the art including heating and solvent evaporation, volatile solvent evaporation followed by lyophilization etc.

In some embodiments and aspects of the invention, the process, or a step thereof, is operated in a reactor, in a batch or in continuous modes.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### General

As used herein the term “about” refers to ±10%.

The terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, and material arts.

As used herein, the term “treating” includes abrogating, substantially inhibiting, slowing or reversing the progression of a condition, of aesthetical symptoms of a condition.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples which, together with the above descriptions, illustrate the invention in a non-limiting fashion.

Materials and Methods

Materials and Methods of Preparation the “Trap”-Oil Emulsion (the “Capsule”)

Microcrystalline cellulose (MC) powder (Avicel®), with a particle size in the range of 20-160 μm, nominal molecular weight (given by the supplier) ˜4.6×104 g/mol (degree of polymerization: 295) was purchased from Sigma-Aldrich Co., Israel. Sodium hydroxide, pentane, and hexane were purchased from Merck Chemicals, Israel.

Samples Preparation

In exemplary procedures, the dissolution was achieved by mixing microcrystalline cellulose (4% wt.) in aqueous sodium hydroxide (7% wt.) for 5 minutes at room temperature and followed by a cooling in a bath (−20° C.) for 10 minutes, using a mechanical stirrer (500 RPM), until no visual crystalline particulates could be observed by light microscopy as measured by wide angle X-ray scattering (WAXS). The solutions were coagulated by addition of deionized water.

The cellulose hydrogel product was rinsed with deionized water to remove alkali traces until the electrical conductivity of the wash water was below 1 mS/cm. The hydrogel content was then determined gravimetrically.

The cellulose/oil emulsions were fabricated in two stages. First, the pre-emulsion was obtained by mixing the cellulose hydrogel dispersion with oil using a high-performance dispersing instrument (IKA®T-18 Ultra-Turrax®, IKA Works Inc., US) at 18000 RPM for 5 minutes, and then the coarse emulsions further were emulsified using a microfluidizer (Model LM-20, Microfluidics, USA). Emulsification was performed by circulating the sample in an air driven microfluidizer through 100 μm Z channel for 4 minutes.

In exemplary procedures, the typical pressure of the liquid jets flowing through the channel was about 140 MPa and the temperature was kept around 50° C. using ice. Typically, the cellulose material to oil ratio (v/v) ranged from 1:1.5 to 1:30, respectively.

Alternatively to direct emulsification of amorphous cellulose, in exemplary procedures, oil drops, coated by cellulose-depleted layer, formed the disclosed capsules spontaneously at the end of the enzymatic cellulose hydrolysis process in the reaction medium of the technological process for producing a biofuel or biodiesel (see FIG. 1).

Equipment and Characterization Methods

Light microscope images were made using an Olympus BH2 microscope using Achromat positive low phase contrast objectives (Olympus, Tokyo, Japan). Images were taken with a 12-bit cooled CCD camera (Sensicam PCO, Kelheim, Germany). Image analysis and measurements were performed using an image analysis software package (ImageJ, National Institute of Health, Bethesda, Md.). Particle size was measured by dynamic light scattering (DLS) with a Mastersizer 2000 instrument (Malvern Co. Ltd., UK).

### Example 1

### The “Trap”-Emulsion Enzyme Recycling Process Description

In exemplary procedures, the recycling process was performed in a biofuel manufacturing process according to the following steps:

### A) The Mixing Step

In exemplary procedures, wastewater stream containing cellulose hydrolysis enzymes (cellulase) was mixed with the disclosed trap-emulsion (i.e. the capsule of the invention, in an embodiment hereof) for 1-5 min in the ratio ranging from 0.01 to 0.15 kg of trap-emulsion per 1 kg of processed cellulose, according to enzymes content in the waste.

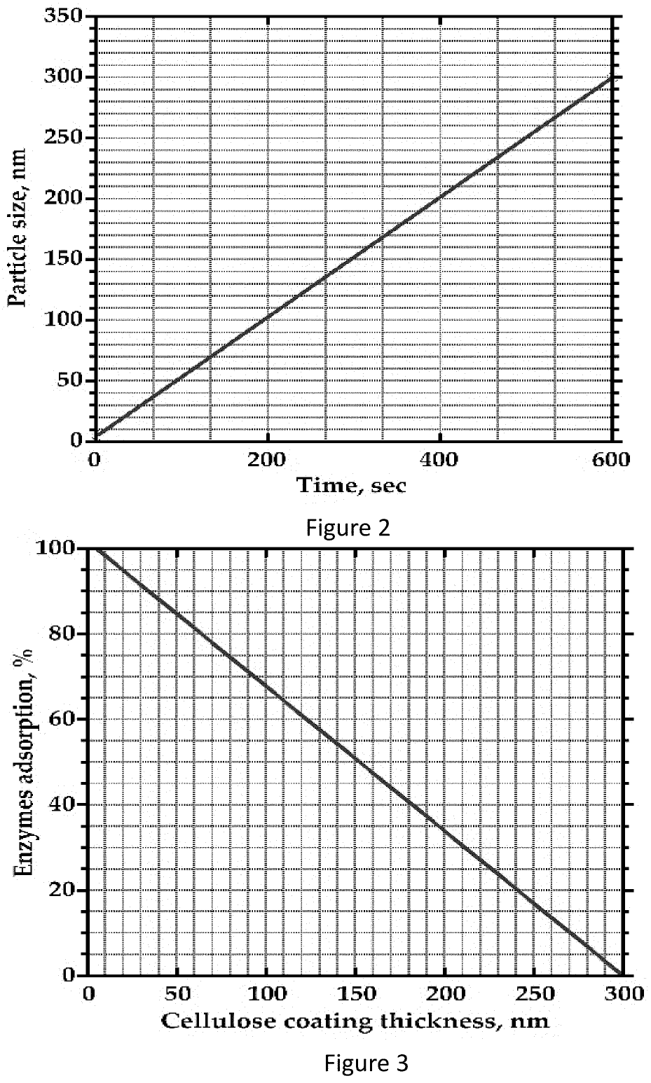

The effectiveness of adsorption of enzymes on the capsules was dependent on the morphology of the “trap” dispersion capsules, see FIG. 2 and FIG. 3.

### B) The Separation Step

In exemplary procedures, 80-100% of the enzymes contained in the wastewater (usually less than 15% of the total volume of processed wastewater) were separated in the settler (during about 1-10 minutes).

### C) The Evaporation Step

In exemplary procedures, evaporation of the volatile hydrophobic substance constituted the trap-emulsion droplets cores (for example, this core comprised low-molecular alkanes: pentane, hexane or even liquefied household gas) was performed under mild conditions (temperature 40-50° C. under vacuum, which does not destroy enzymes);

### D) Releasing the Absorbed Enzymes

In exemplary procedures, the absorbed enzymes were released into aqueous surrounding medium accompanied by the enzymatic hydrolysis of cellulose residues of the former emulsion shell.

After the previously described operations the activity of recycled enzymes remains 80-100% of the initial value.

### E) Recycling

In exemplary procedures, the resulting aqueous solution comprising active regenerated enzymes and a small amount of dissolved fermentable sugars (derived from cellulose shells hydrolysis at the previous process stage) was directly returned to the main biofuel manufacturing process.

It was found that the disclosed trap-emulsion can adsorb from the wastewater 80-100% of all different cellulases contained in standard industrially used enzyme cocktails, for example, Novozym® 1.5L or Accelerase® 1500, after which all adsorbed enzymes retained their activity at their release during the destruction of the trap-emulsion drops.

Taken together, it can be concluded, that the recycling process necessitated recycling only 1-15% vol. of the main wastewater stream. Without being bound by any particular theory or mechanism, it is assumed that the usage of the relatively small amount of volatile oil as the core, contained in the trap-emulsion, allows to significantly lower the energy consumption for evaporation in this process, compared with e.g., the evaporation of the same amount of water.

Hence, the minimal carrier-oil regenerative losses and degradation, the reversibility of the disclosed process, as well as the absence of chemical modification of enzymes and costly membrane filtration processes, makes the use of the herein disclosed trap-emulsions attractive for enzymatic regeneration in the biorefinery industry.

An additional phenomenon discovered was the fact that if the internal dispersed contents were removed out of the cellulose coating (previously saturated with these absorbed enzyme), these adsorbed enzymes could be fully released into the external aqueous environment and their activity completely restored. Results of a non-limiting example of determining enzymatic activity using an emulsion compared to a hydrogel after a first and a second recycling round is shown under Table 1, below.

TABLE 1

| | Degree of | Degree of |
|---|---|---|
| | emulsion | hydrogel |
| | hydrolysis | hydrolysis |
| Initial enzymatic hydrolysis process: | ~96% | ~96% |
| After a first enzyme recycling round: | ~93% | ~93% |
| After a second enzyme recycling round: | ~39% | ~73% |

The removal of the above internal core of the dispersion were performed by means of centrifugation, melting or by means of evaporation of this core, in accordance with the overall nature of the composition. The overall process was organized in such a way that the properties of the enzyme were not affected.

A non-limiting example of process steps utilizing the novel trap-emulsion to recover cellulose hydrolysis enzymes from process waste stream and the probing of the functionality of the recovered enzymes is shown in the scheme shown in FIG. 1.

Further, the addition of trap-emulsion to the aqueous suspension of solid residues (i.e. unhydrolyzed cellulose/lignin, which adsorbs/captures enzymes, even if they cannot hydrolyze it) and enzymes (in ratios 1:3-1:1 of the residues with respect to the mass of adsorbed enzymes) allowed the extraction of adsorbed enzymes from the solid residues due to competing adsorption between the trap-emulsion and solid residues.

This effect provides the possibility of recycling at least 50% wt. of these absorbed enzymes to the main cellulose hydrolysis process.

Further, the residues of the cellulose emulsion shells did not reduce the efficiency of the enzyme regeneration process. On the contrary, these remnants were subjected to enzymatic hydrolysis, being completely and very quickly hydrolyzed to glucose by the enzymes released from the trap-emulsion, bringing the additional 0.1-3% wt. of fermentable sugars into the main biofuel manufacturing process.

### Example 2

### Microscopic Examination of the Capsules

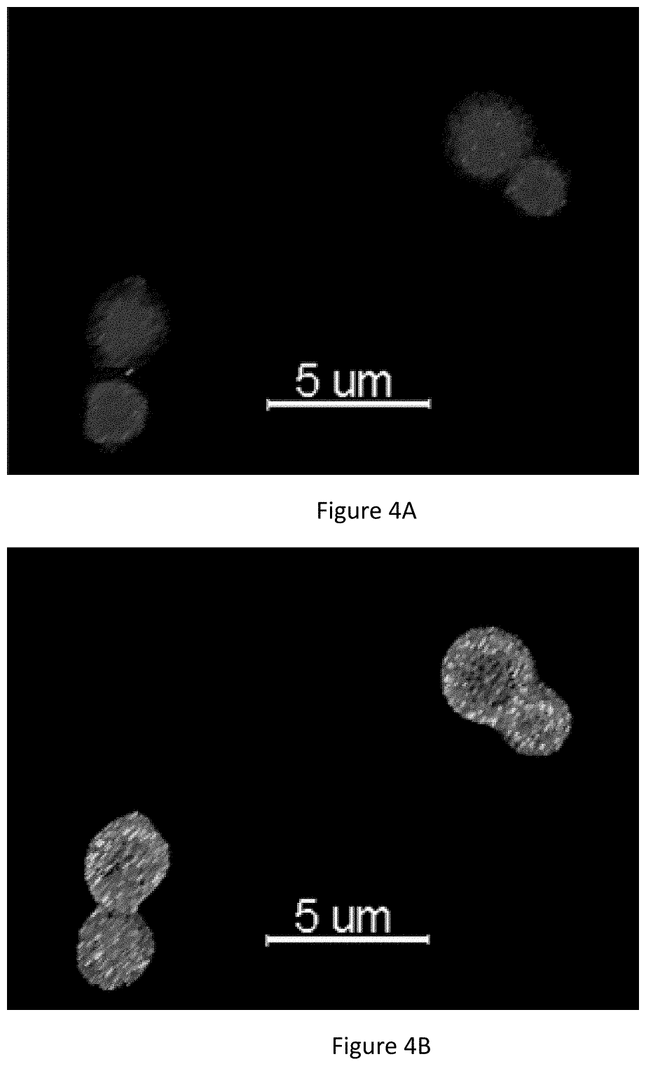

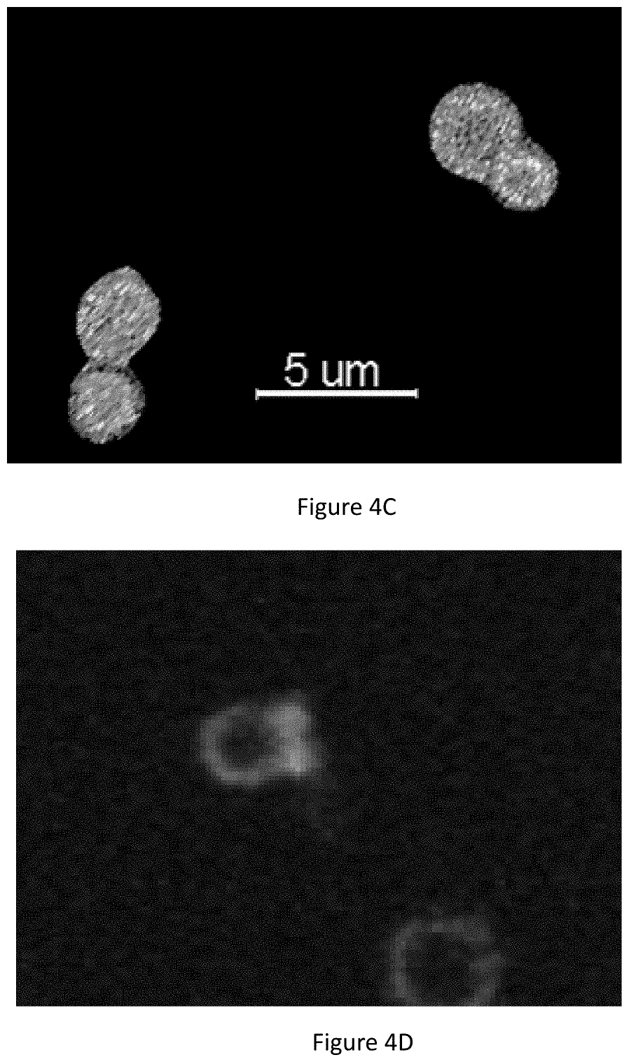

FIGS. 4A-C present fluorescent microscope images of cellulose-coated oil emulsion at the beginning and at the end of the enzymatic cellulose hydrolysis process (FIGS. 4C and 4D, respectively). At the beginning, the “trap”-emulsion formation (the capsules, comprising both oil and cellulose) has a visible uniform cellulose casting and smooth boundary between the cellulosic shell and oil core.

These capsules were suitable for use as a trap for the isolation of enzymes from processing wastewater for their further regeneration and return to the reaction medium of the technological process.

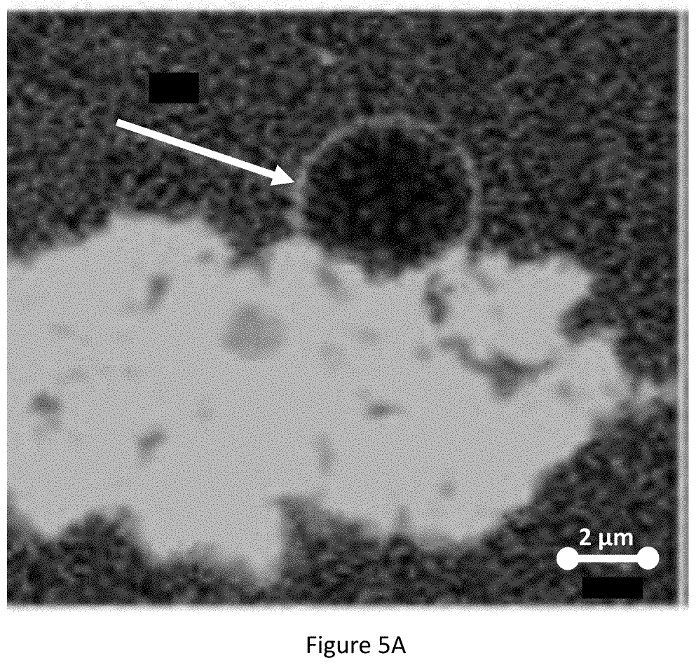

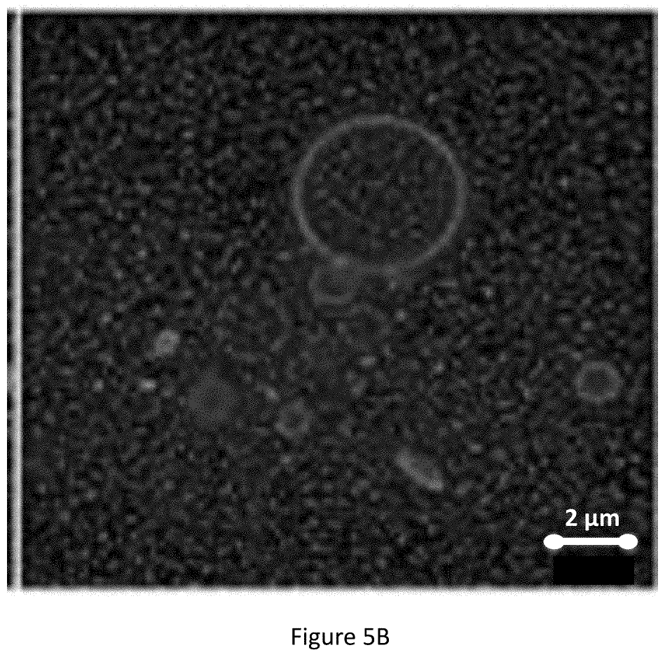

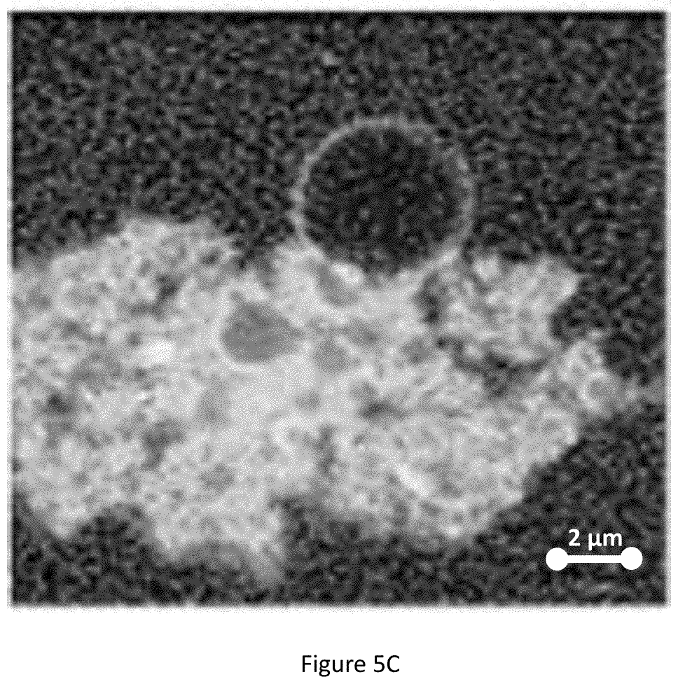

FIGS. 5A-C show fluorescent microscope images of the “trap”-oil emulsion drop's shell, consisting of thin cellulose-depleted/oil-remnants mixture, saturated with captured enzymes (cellulose is colored by cellulose-specific fluorescent pigment (green color); oil—by the oil-specific fluorescent pigment (red color).

The protein (unadsorbed enzymes) content in the aqueous solutions, was evaluated with the Bradford method, known in the art, showing evidence of complete removal of enzymes from their aqueous solutions by their adsorption on the “trap”-emulsion drops.

It is noteworthy that in order to imitate the distillation stillage and wastewaters of existing biorefinery enterprises containing the used enzymes, only minimal amounts of the disclosed “trap”-emulsion were added to the liquids.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A composition comprising at least one capsule comprising a shell and a hydrophobic core, wherein:
(a) a median diameter of said capsule is less than about 3000 nm;
(b) the shell comprises at least one layer comprising cellulosic material, said layer having a thickness of 5 to 50 nm; and
(c) the shell is characterized by an average porosity of 85 to 99%.

2. The composition of claim 1, wherein said cellulosic material is in the form selected from amorphized non-derivatized cellulose, an assemblage of cellulosic particles, and a combination thereof.

3. The composition of claim 1, wherein the porosity ranges from 94% to 96%.

4. The composition of claim 1, wherein the weight ratio between said hydrophobic core and said at least one cellulose layer ranges from 30:1 to 1.5:1, respectively.

5. The composition of claim 1, wherein the hydrophobic core comprises one or more materials selected from C2-C10 alkanes, alkenes, alkynes, substituted or non-substituted, and silicones, and any combination thereof.

6. A composition comprising at least one capsule comprising: (i) a shell in the form selected from amorphized non-derivatized cellulose, an assemblage of cellulosic particles, and a combination thereof, (ii) a hydrophobic core, and (iii) one or more hydrolyzing enzymes, wherein:
(a) a median diameter of said capsule is less than about 3000 nm;
(b) the shell comprises at least one layer, wherein said layer having a thickness of 5 to 300 nm;
(c) said hydrolyzing enzymes are adsorbed on the shell.

7. The composition of claim 6, wherein the enzymatic activity of said adsorbed hydrolyzing enzymes in the hydrophilic core medium is less than 20% compared to the enzymatic activity of the corresponding non-adsorbed hydrolyzing enzymes in said hydrophilic medium.

8. The composition of claim 6, wherein said one or more hydrolyzing enzymes are selected from the group consisting of: α-amylase, β-amylase, isoamylase, glucoamylase, pullulanase, cyclodextrin glucano-transferase, β-fructofuranosidase, glucose isomerase, glycoside hydrolase, and combinations thereof.

9. The composition of claim 6, wherein the said one or more hydrolyzing enzymes and said shell are present in a weight ratio of 1:50 to 1:5, respectively.

10. The composition of claim 9, wherein the weight ratio is 1:40 to 1:30, respectively.

11. (canceled)

12. A method for recycling one or more cellulose hydrolyzing enzymes into a reaction liquid, the method comprising:
(a) contacting at least a portion of the reaction liquid comprising the cellulose hydrolyzing enzymes to be recycled with at least one capsule comprising a shell and a hydrophobic core;
(b) eliminating said hydrophobic core so as to disintegrate the capsule, thereby desorbing the hydrolyzing enzymes from the shell;
(c) recycling the released enzymes into the reaction liquid;
wherein:
(i) a median diameter of said capsule is less than about 3000 nm; and wherein (ii) the shell comprises at least one layer comprising cellulosic material, wherein said layer has a thickness of 5 to 300 nm.

13. The method of claim 12, further comprising a step of contacting remnants of the eliminated hydrophobic core with amorphized cellulose, thereby re-forming the capsule.

14. The method of claim 12, wherein said thickness ranges from 5 to 50 nm.

15. The method of claim 12, wherein the shell is characterized by an average porosity of 85 to 99%.

16. The method of claim 12, wherein the cellulosic material is in the form selected from amorphized non-derivatized cellulose, an assemblage of cellulosic particles, and a combination thereof.

17. The method of claim 12, wherein the step of eliminating said core is performed by evaporating said hydrophobic medium.

18. The method of claim 12, wherein said released enzymes are characterized by an enzymatic activity of at least 50% of their activity prior to adsorption.

19. The method of claim 16, further comprising a step of hydrolyzing remnants of the said layer comprising the amorphized non-derivatized cellulose or the assemblage of cellulosic particles in the reaction liquid.

20. The method of claim 12, being employed in the production of biofuel or biodiesel.

21. The method of claim 20, wherein the capsule is formed spontaneously in the reaction liquid, simultaneously with the production of biofuel or biodiesel.
